(19) United States
(12) Patent Application Publication
(10) Pub. No.: US 2012/0041496 A1
(43) Pub. Date: Feb. 16, 2012

(54) Swivel Screw Ligament Fixation Device

(76) Inventor: Peter Michael Sutherland Walker, New South Wales (AU)

(21) Appl. No.: 13/265,130

(22) PCT Filed: Apr. 20, 2010
(86) PCT No.: PCT/AU2010/000446

§ 371 Date: Oct. 18, 2011

(30) Foreign Application Priority Data
Apr. 20, 2009 (AU) 2009901673

(51) Int. Cl. A61B 17/86

(52) U.S. Cl. 606/321

(57) ABSTRACT

A surgical screw fastener device and method for pulling a graft through a first tunnel defined in a first bone. The surgical screw fastener including a body having a proximal end and a distal end, and an exterior screw thread located around the body for threadedly engaging a wall of the first tunnel. The device can have a first coupling element at the proximal end of the body, wherein the first coupling element adapted to couple a driver tool. The device can further have a second coupling element for rotatable coupling of a first end of the graft with respect to the device.

### FIELD OF THE INVENTION

The present invention relates to fastening devices and in particular to screw fastening devices.

The invention has been developed primarily for use as a fastener that can be used from within a bone tunnel to pull a graft (including tendons or ligament) though a tunnel (or hole) in a bone and will be described hereinafter with reference to this application. However, it will be appreciated that the invention is not limited to this particular field of use.

### BACKGROUND OF THE INVENTION

Any discussion of the prior art throughout the specification should in no way be considered as an admission that such prior art is widely known or forms part of the common general knowledge in the field.

The present disclosure will be used with reference to an anterior cruciate ligament (“ACL”) reconstruction, but it will be understood that the technology and methods of the present invention may have other applications.

The ACL reconstruction can be done in numerous ways. All common methods involved drilling holes or tunnels in the femur and tibia. These can be drilled from any direction using a variety of techniques. Grafts such as autografts, allografts or artificial biomaterials may be used to extend between the femoral tunnel and the tibial tunnel. The graft is then fixed to the appropriate bone structure, again numerous techniques being suitable. The replacement graft is fixed to the femur and tibia, most commonly by a screw into the adjacent bone, it being understood that staples, pins and similar devices may also be used. In general, and most commonly, the graft is tensioned prior to finally affixing it to the bone. Devices are known that can exert tension onto the graft before affixation to the bone takes place. These tension devices hold the graft in tension while fixing screws are inserted to fasten the graft in place. Therefore, this tension device can not fine tune or adjust the tension of the graft once the remaining lose end is fixed in place.

### Object of the Invention

It is an object of the present invention to overcome or ameliorate at least one of the disadvantages of the prior art, or to provide a useful alternative.

It is an object of the invention in its preferred form to provide a device for tensioning a graft in an ACL reconstruction.

It is an object of the invention in its preferred form to provide a device and methods for adjusting graft tension, after both ends are affixed to the bone.

### SUMMARY OF THE INVENTION

According to an aspect of the invention there is provided a surgical screw fastener device for pulling a graft through a first tunnel defined in a first bone, the surgical screw fastener including:
a body having a proximal end and a distal end;
an exterior screw thread located around the body for threadedly engaging a wall of the first tunnel;
a first coupling element at the proximal end of the body, the first coupling element adapted to couple a driver tool; and
a second coupling element for rotatable coupling a first end of the graft with respect to the device.

Preferably, the passage is a through passage having an aperture at the distal end and the proximal end.

Preferably, the first coupling element is a female socket at the proximal end of the body. More preferably, the passage is a through passage having an aperture at the proximal end that defines the socket.

Preferably, the first coupling element is a releasable coupling element for securely coupling the device to the driver tool.

Preferably, the passage has an aperture at the distal end; and the a second coupling element comprises a saddle element that is locatable within the passage and is adapted to be rotatable with the passage. More preferably, the saddle element is locatable within the passage by being passed through an aperture at the proximal end defined by the passage. Most preferably, the saddle element is adapted to rotate freely within the passageway and is restrained in its axial movement toward the distal end by a necking down of the passageway.

Preferably, the second coupling element comprises a saddle element locatable within the passage, and is adapted to be rotatable with the passage; and the a second coupling element further comprises a fastening element coupled to the saddle element and adapted to retain a first end of the graft. Preferably, the fastening element is integrally formed with the saddle element. Preferably, the fastening element is integrally formed with an artificial graft. Preferably, the fastening element is constructed of a flexible material.

Preferably, the fastening element includes any one or more of the set comprising: a loop element; a net element.

Preferably, selective clockwise or anticlockwise rotation of the body, while threadedly engaging the wall of the first tunnel, can respectively increase or decrease tension applied to the graft. Alternatively, selective anticlockwise or clockwise rotation of the body, while threadedly engaging the wall of the first tunnel, preferably respectively increase or decrease tension applied to the graft.

According to an aspect of the invention there is provided a method of using a surgical screw fastener device for pulling a graft through a first tunnel defined in a first bone, the method comprising the steps of:
(a) providing a screw fastener device;
(b) coupling the screw fastener to a driver tool passed through the first tunnel;
(c) rotatably coupling a first end of the graft to the device;
(d) rotating the screw fastener, by rotating the driver tool, causing the screw fastener to threadedly engage the bone and thereby draw the graft up though the tunnel;
(e) with other end of the graft fixed in location, the screw fastener can be rotated with respect to the bone to thereby set a tension applied to the graft; and
(f) detaching the screw fastener from the driver tool.

Preferably, the screw fastener device is as herein described.

Preferably, fixing the other end of the graft includes abutment of an end plug fixed to the other end of the graft as a result of the device drawing up the graft.

According to an aspect of the invention there is provided a screw fastener having a longitudinal through passageway. One end of the passageway comprises a socket for receiving a fastener driver tool (or adaptor). The other end of the passageway provides a portal for the looped material or artificial graft.

A saddle is preferably located within the passageway. The saddle is not able to pass through the loop portal. The saddle is adapted to receive a suture, suture loop or a loop of material through which the graft is placed. More preferably, the saddle is able to rotate within the passageway when the suture and/or loop is in tension.

A saddle is preferably located within the passage but not able to pass through the loop portal. More preferably the saddle can rotate within the passage. Most preferably, with the screw fastener located within a tunnel (or hole) formed in a bone, the saddle enables the screw fastener to be rotated with respect to the bone without the tendon undergoing a corresponding rotation.

Preferably a screw fastener can be located within a tunnel (or hole) formed in a bone and rotatably coupled to a graft, wherein rotation of the screw fastener with respect to the bone pulls the graft though the tunnel. More preferably, rotation of screw fastener enables controlled pulling of a graft up though a tunnel. Most preferably, with each end of the graft coupled to a respective bone, rotation of screw fastener in one or another direction enables a respective increased and decreased tensioning of the graft.

In preferred embodiments the fastener has a pair of transverse openings, each leading to a longitudinal, external channel. The transverse openings in the channels are preferably adapted to receive a fastener such that the driver can be rotated in either direction. A lip on the inside of the screw prevents it from being pulled out of the screw. An alternative technique to couple the driver to the screw would be to have holes and a form of attaching suture to the screw for tying it to the driver either in a slot on the side of the driver or through the middle of the driver if cannulated.

A screw fastener preferably includes a locking mechanism that can pull the screw, in tension, thus into place and then be unlocked once the screw has the correct tension.

Preferably the driver is adapted to provide an indication of the torque on the fastener.

Preferably, screw fastener can be pulled into position by a driver. More preferably, the driver can be released once it screw fastener is in position.

Preferably a graft includes any one or more of the set comprising a transplant tendon, an artificial tendon and a transplant ligament, and an artificial ligaments.

### BRIEF DESCRIPTION OF THE DRAWING FIGURES

In order that the invention be better understood, reference is now made to the following drawing figures in which:

FIG. 1A is an underside perspective view of an embodiment fastener made in accordance with the teachings of the present invention;

FIG. 1B is a top perspective view of the fastener depicted in FIG. 1A;

FIG. 2 is a side elevation of the fastener depicted in FIG. 1A;

FIG. 3 is a cross section through line 3-3 of FIG. 2;

FIG. 4 is a perspective view of a saddle;

FIG. 5 is a side elevation view of an embodiment fastener with the saddle retaining a suture loop that passes through the portal;

FIG. 6 is a cross sectional view through line 6-6 of FIG. 5;

FIG. 7A-7K are perspective views illustrating steps involved in utilisation of the invention in conjunction with an ACL reconstruction;

FIG. 8A is a perspective view of another embodiment form of driver and driver engagement;

FIG. 8B is an enlarged perspective view of FIG. 8A, showing detail of the driver engagement;

FIG. 8C is a cross sectional view of an embodiment driver and engagement of FIG. 8A;

FIG. 8D is a side elevation of the device depicted in FIG. 8A;

FIG. 9A is a perspective view of a further embodiment of a driver;

FIG. 9B is a cross section and detail of the driver depicted in FIG. 9A, illustrating the engagement;

FIG. 9C is an enlarged cross section of FIG. 9B, showing detail of the driver engagement;

FIG. 10A is a perspective view of an embodiment screw fastener made in accordance with the teachings of the present invention;

FIG. 10B is a sectional view of the screw fastener of FIG. 10A;

FIG. 11A is a perspective view of an embodiment screw fastener made in accordance with the teachings of the present invention;

FIG. 11B is a sectional view of the screw fastener of FIG. 11A;

FIG. 12A is a side elevation view of an embodiment driver;

FIG. 12B is an enlarged partial side elevation view of the screw fastener engagement of the driver of FIG. 12A;

FIG. 12C is an enlarged end elevation view of the driver of FIG. 12A;

FIG. 13A is a side elevation view of an embodiment driver;

FIG. 13B is an enlarged sectional end elevation view of the screw fastener engagement of the driver of FIG. 13A, shown engaged with a screw fastener;

FIG. 14A is a sectional view of an embodiment fastener made in accordance with the teachings of the present invention;

FIG. 14B is a sectional view of an embodiment fastener made in accordance with the teachings of the present invention;

FIG. 14C is a sectional view of an embodiment fastener made in accordance with the teachings of the present invention;

FIG. 14D is a perspective view of an embodiment fastener made in accordance with the teachings of the present invention;

FIG. 15A-15D are perspective views illustrating steps involved in utilisation of the invention in conjunction with an ACL reconstruction; and

FIG. 16 is a flowchart for an embodiment method of a screw fastener in conjunction with an ACL reconstruction.

### BEST MODE AND OTHER EMBODIMENTS

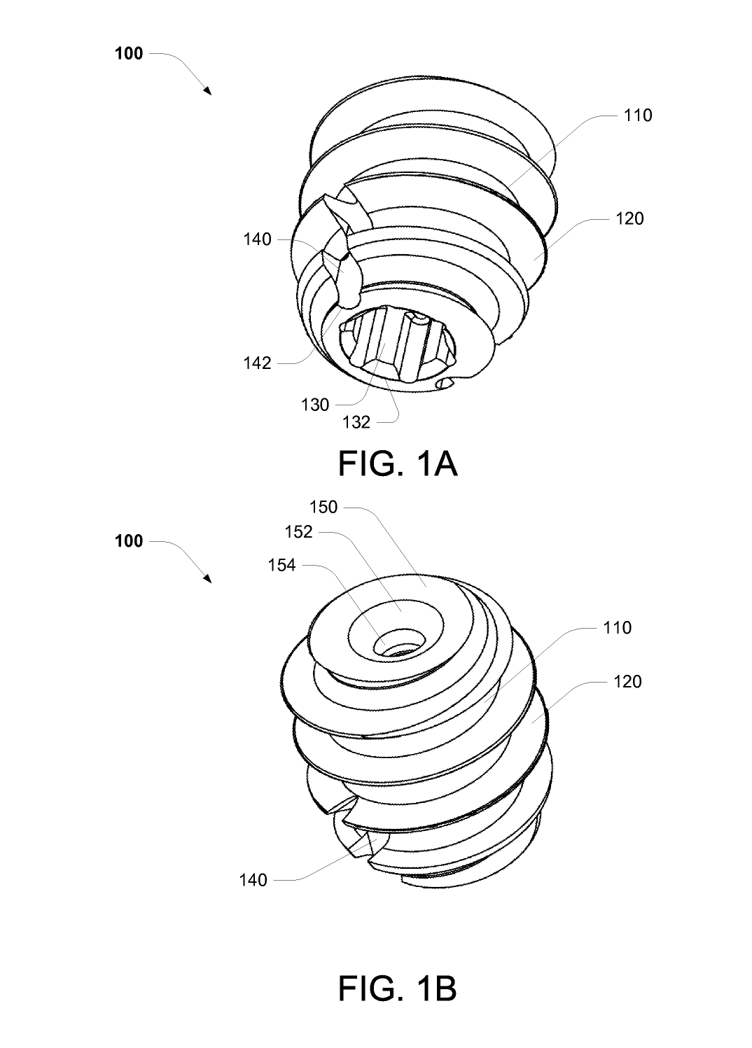

As shown in FIG. 1A, a fastener 100, in accordance with the teachings of the present invention, comprises a relatively coarse threaded slightly tapered plug (or body) 110 having the fastening characteristics of a bone screw. External screw threads 120 are adapted to be self tapping into a tunnel pre-drilled through the tibia or femur. A central longitudinal bore or passageway 130 extends through the fastener, from one end to the other.

By way of example, a device of this kind will typically be about 6 mm to 14 mm in diameter and have a length of about 15 mm to 20 mm.

In an embodiment, a proximal end of the passageway 132 forms a socket for receiving a driver (including a adaptor and/or tool) such as a Torx brand driver. It will be appreciated that the socket, and therefore the head of a corresponding driver, can comprise numerous configurations, including a hex socket, and/or a star socket. A driver is adapted to accommodate the socket configuration.

Opposing longitudinal channels 140 extend approximately a third to halfway down the body of the fastener, into the screw threads, providing a relief groove that starts by intersection of the proximal rim 142 of the fastener and terminates at one of a pair of transverse through openings (not shown in this view). The channel 140 interrupts the screw threads and the proximal rim providing a space that can accommodate a loop of material such as polyethylene or polyester or other type of suture material without interfering with the operation of the fastener, the fastener's threads or the socket 130. In this embodiment channels 140 are provided to enable the screw to facilitate more aggressive cutting engagement with the bone.

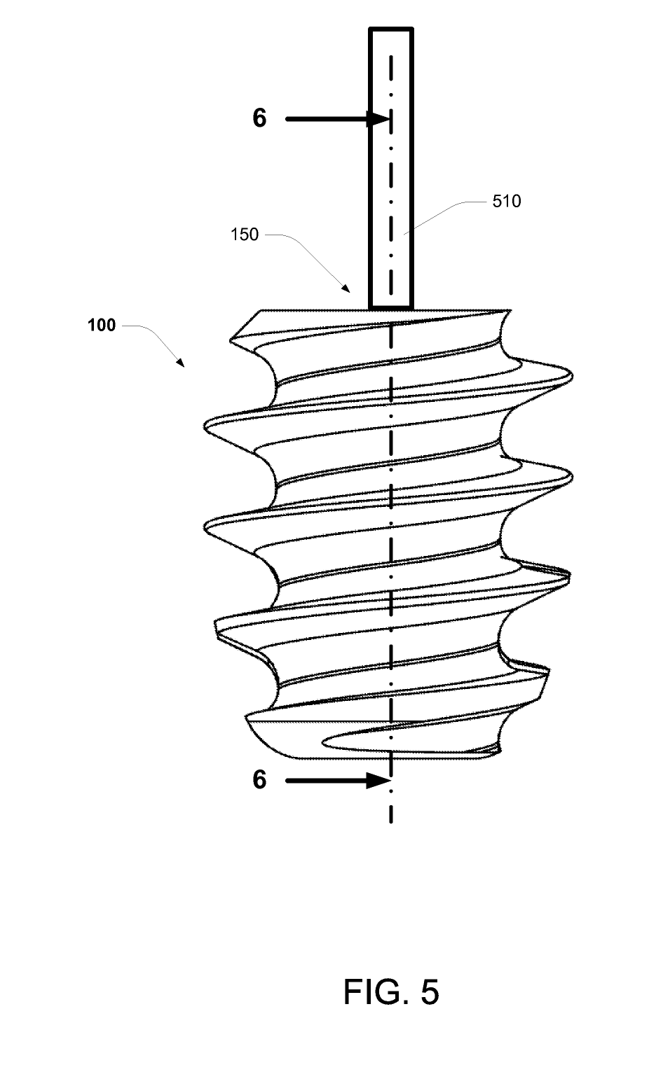

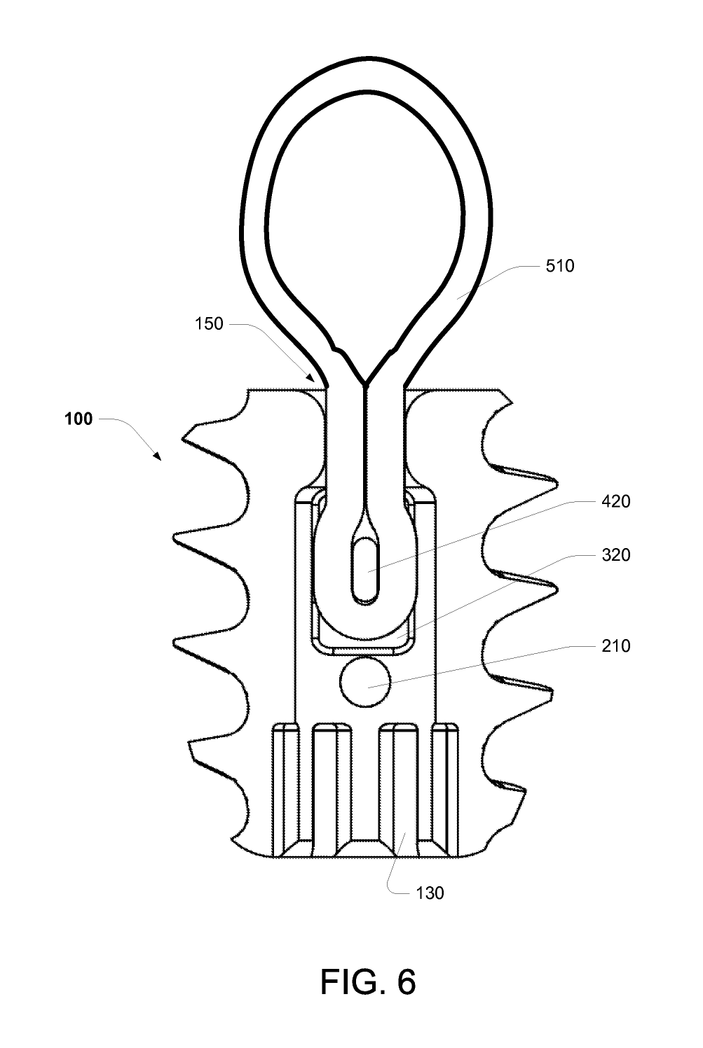

As shown in FIG. 1B, the distal end 150 of the fastener 100 comprises a portal 152 that leads into the central bore or through passageway 130. The portal 152 comprises a smoothly radiused rim and a smooth opening 154 for receiving the flexible loop or suture arrangement that will be described with reference to FIG. 5 and FIG. 6.

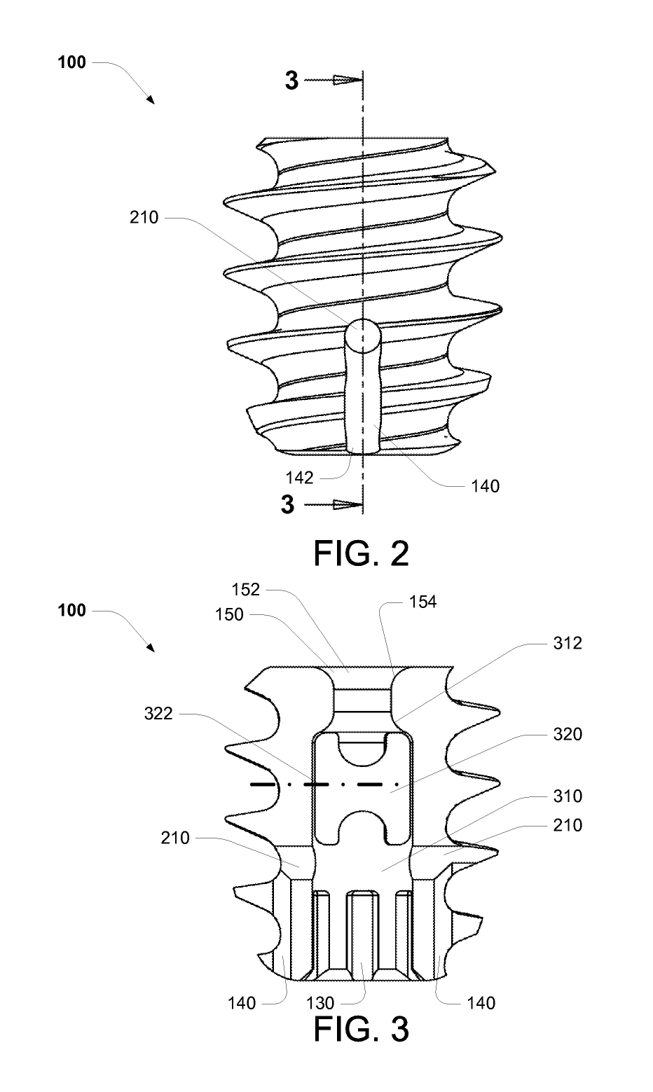

As shown in FIG. 2 and FIG. 3, each longitudinal, external channel 140 terminates in a transverse through opening. The through openings lead into the central passageway 310. The longitudinal channel and transverse channels can be used for engaging and/or locking a cooperating pulling device or driver.

A saddle 320 is adapted to rotate freely within the passageway and is restrained in its axial movement toward the portal 152 by a necking down 312 of the passageway adjacent to the portal 152. Saddle is not able to pass through the loop portal due to a lip on the inside of the screw fastener. The saddle rotates within the screw as a graft is being pulled up so as to not twist the graft (or tendon or ligament).

It will be appreciated that, references to a graft includes a transplant or artificial tendons and/or ligaments.

As shown in FIG. 3, the central passageway 310 is adapted to receive a saddle 320 between the openings 210 and the necking 312. In preferred embodiments, the saddle is symmetrical about its transverse axis 322 so that it may be inserted into the passageway and used in either orientation. The edges of the longitudinal ends 324, 326 are radiused to cooperate with the necking 312, thus reducing friction. Note that the through openings 210 are formed beyond the axial reach of the socket 130 so that the suture that passes through the openings 210 does not interfere with the head of the driver.

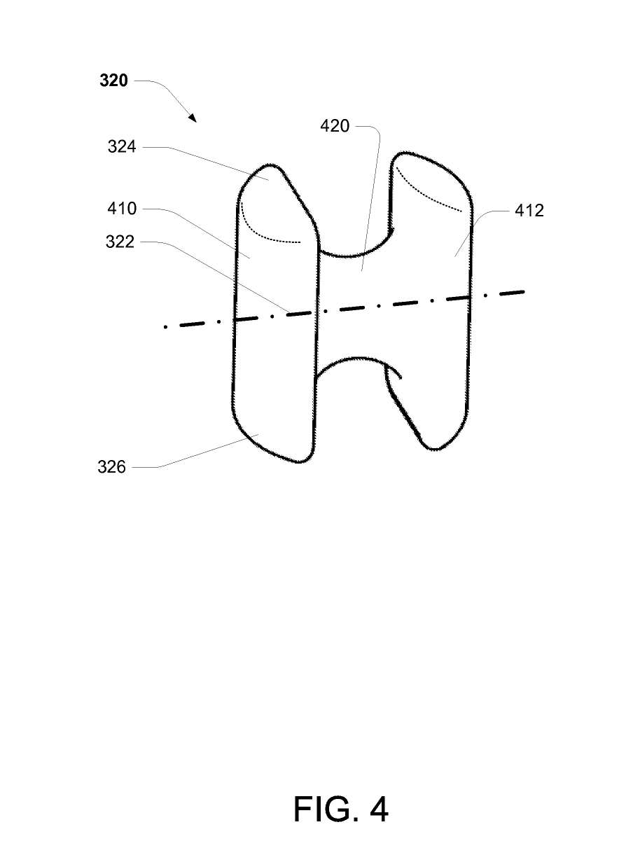

As shown in FIG. 4, the saddle 320 is generally “H” shaped, but can be of other shapes. The lateral components 410, 412 are essentially sections of cylinder and are joined together by a smooth integral cross member 420. The cross member 420 is smoothly blended into the interior surfaces of the lateral portions 410, 412. The cross member 420 is necked, providing a minimum diameter in the middle and a gradual flaring toward the lateral members 410, 412.

As shown in FIG. 5 and FIG. 6, a loop of fibre material (for example suture material) 510 may be passed around the cross member 420 of the saddle 320 to form a constrained loop. The loop 510 enters through and exits through the loop portal 152. Note the lack of sharp edges in the area of the portal.

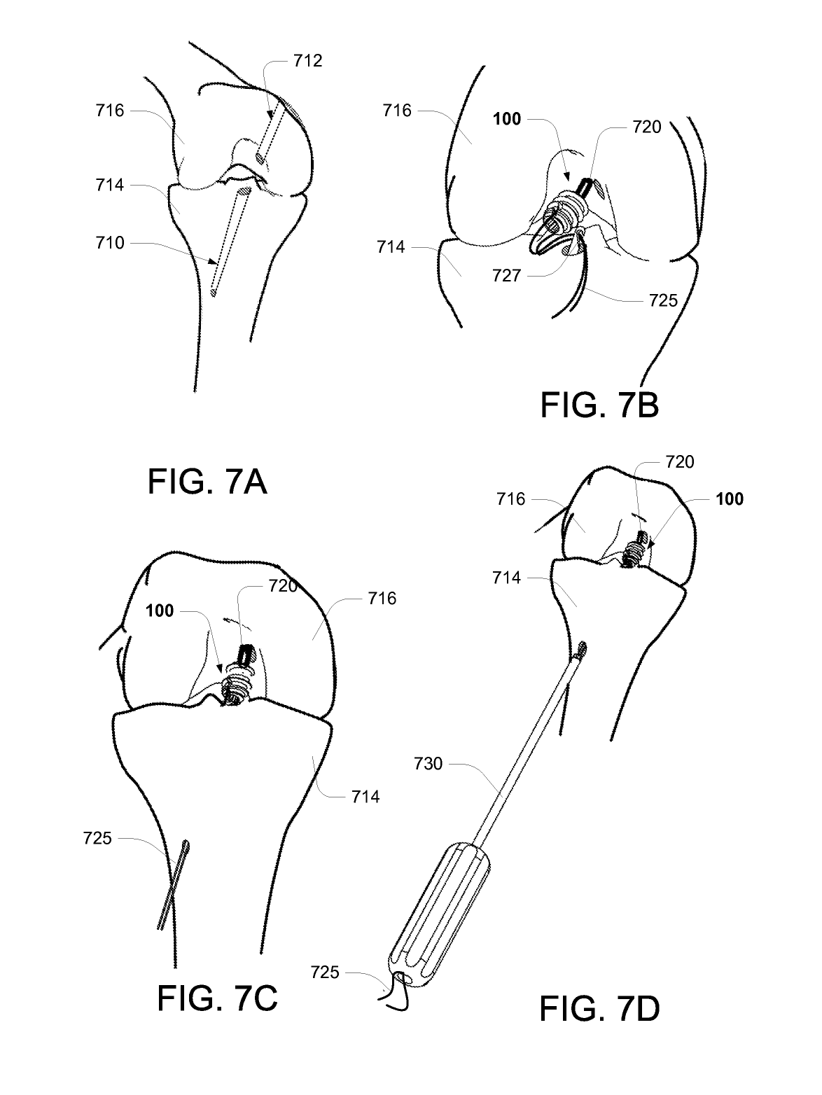

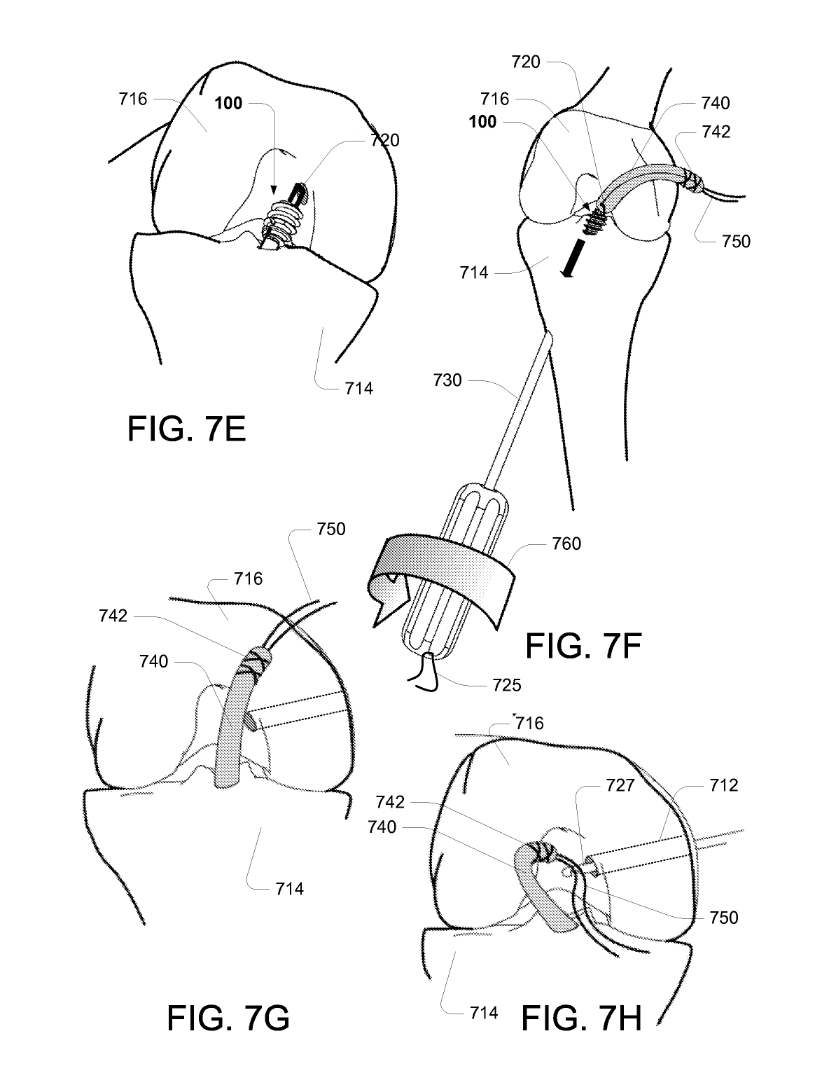

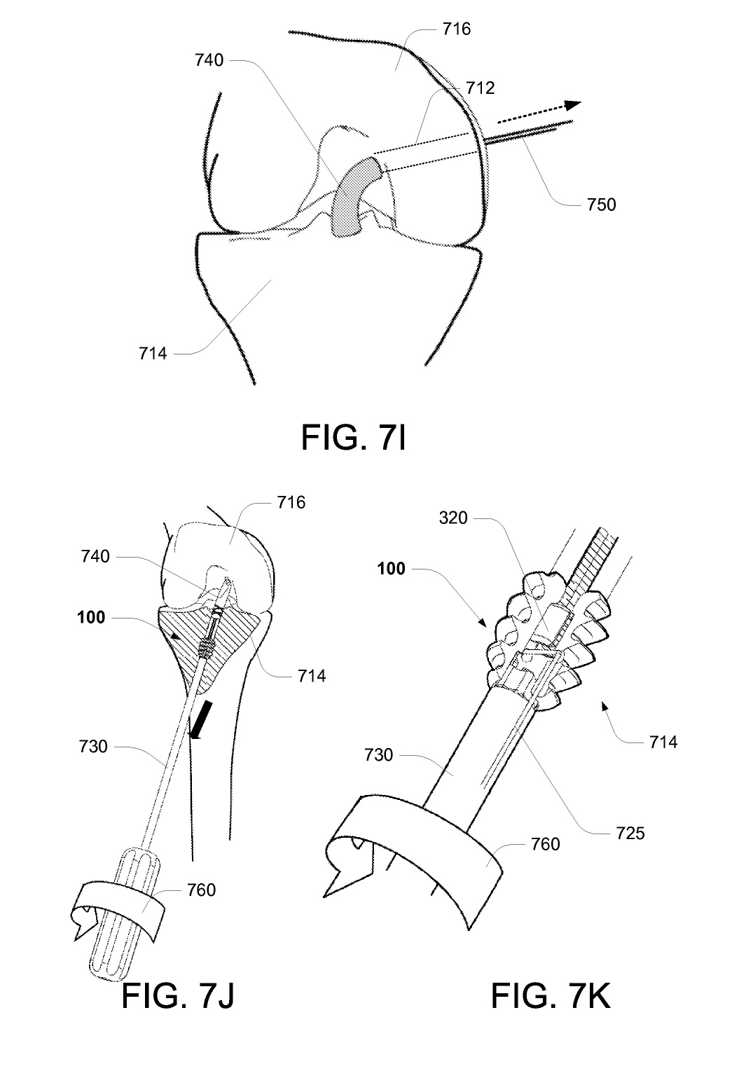

FIG. 7A-FIG. 7K illustrate how the fastener is used in an embodiment method of an ACL reconstruction method. As shown in FIG. 7A, through holes or tunnels 710, 712 are formed through the bones of the tibia 714 and the femur 716. As shown in FIG. 7B a first suture material 720 forms a loop around the saddle as illustrated in FIG. 6. Typically, the first suture material comprises a loop extending around the saddle and adapted to extend below the screw fastener for receiving the graft. A second suture 725 passes through the transverse openings 210. The second suture 725 is collected with a small hook 727 that is inserted through the tibial tunnel 710. The second suture is then withdrawn from the opposite end of the tibial tunnel. In one embodiment, the free end of the second suture 725 is passed e.g. through the head, shaft and handle of an appropriate driver 730, and tightened to the driver. This allows the driver 730 to be advanced through the tibial tunnel 710. As shown in FIG. 7F, the replacement tendon 740 is passed through the loop, whipped or otherwise attached to the first suture material 720. It will be appreciated that the tendon can be placed through the loop before it is inserted into the knee or could be coupled when the tendon is in the knee By way of example, first suture comprises a loop extending around the saddle and adapted to forming a loop below the screw fastener for receive the tendon therethough. The driver 730 is fully inserted into the socket 130. In this position, the second suture 725 is used to attach or temporarily lock the fastener onto the head of the driver 730 by tensioning the free end of the second suture 725. As shown in FIG. 7H a third suture 750 is whip stitched onto the femoral side 742 of the replacement tendon structure 740 and the third suture 750 is picked up with a hook 727 and drawn through the femoral tunnel 712. As shown in FIG. 7I, the femoral end whip stitched suture is then pulled through the femoral tunnel and then affixed with an appropriate device to the femur bone. As suggested by FIG. 7J, the femoral end of the tendon is now fixed to the femur and the tibial end of the tendon is affixed to the saddle within the threaded fastener 100. At this point the driver 730 is rotated anti-clockwise 760, thus retracting the fastener 100 into the tunnel 710 toward the driver 730. It will be appreciated that in an alternative embodiment the screw thread can be configured such that clockwise rotation of the driver causes the screw fastener to be retracted into the tunnel. The retraction of the fastener 100 tensions the tendon and the degree of tension is determined by the extent of rotation and/or torque imposed by the surgeon. Because the saddle 320 rotates freely within the fastener 100, the tendon does not become twisted as it becomes tensioned. When the appropriate tension is reached, the driver 730 and the second suture 725 can be withdrawn from the tibial tunnel 710. This procedure can be done in either direction such that the screw can end up in the femur or tibia.

In an embodiment, a suture is used to fasten a driver to a screw fastener. The suture can be attached to the screw, typically passing through 2 holes in the screw fastener. By way of example, the suture can then be located to the side of the driver, or pass through the middle of a cannulated driver. The suture can be tensioned and tied at the proximal end of the driver to hold the screw in place so it can be pulled/drawn up to a tunnel in a bone. The suture is typically removed once the screw is in placed.

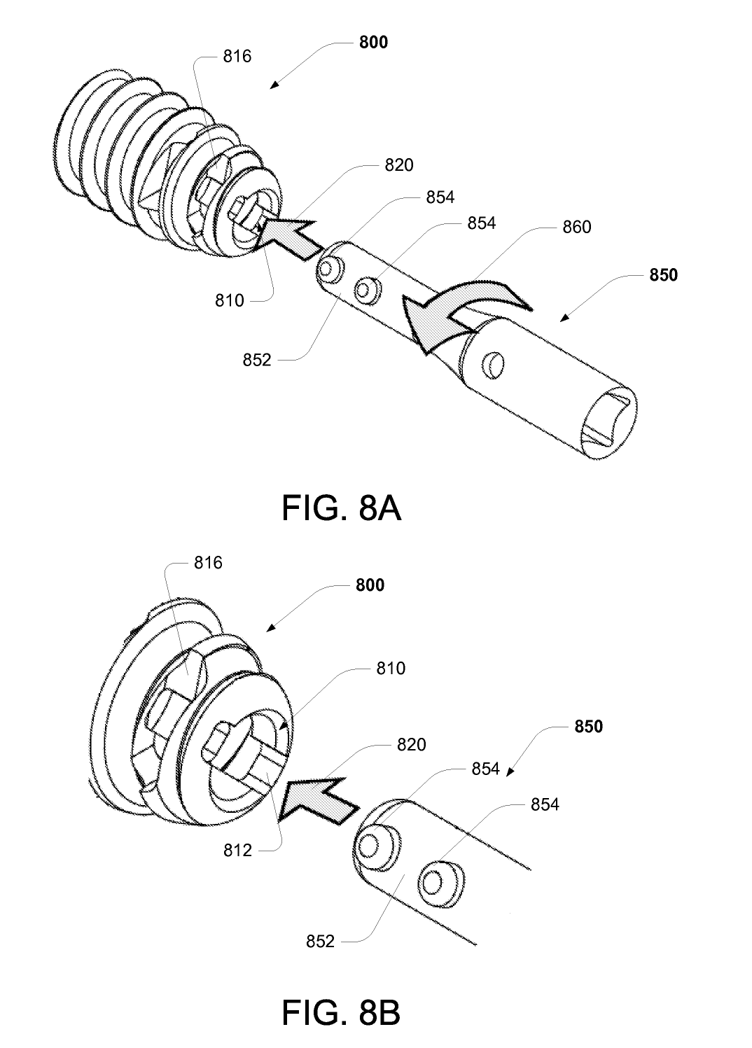

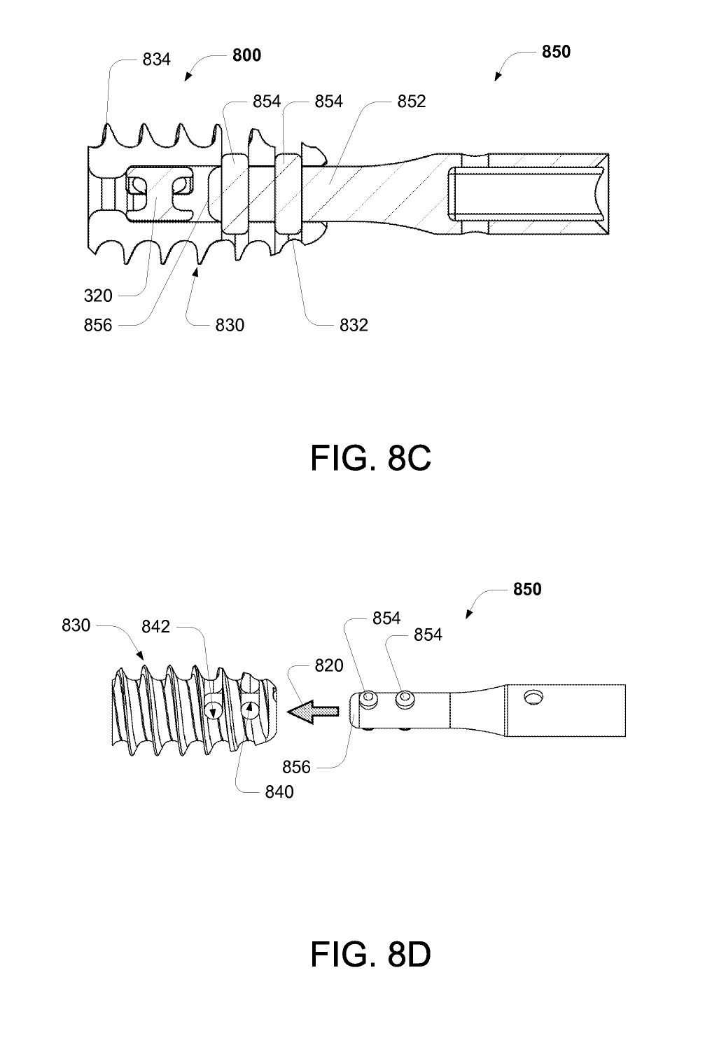

As shown in FIG. 8A and FIG. 8B a fastener 800 has been configured to receive a specially adapted driver 850. The driver 850 comprises a generally cylindrical tip 852 having one or more radially extending pins or projections 854. In this example, the driver 850 is provided with four pins. The pins are provided in adjacent pairs that are diametrically opposed to one another on the tip 852. It will be appreciated that other configurations are contemplated. In particular, in an embodiment only one pin or protrusion by be provided. It will be appreciated that pins can be of any cross section, for example circular, square or rectangular.

In order that the fastener 800 receive the tip of the driver 852, the internal bore 810 of the fastener 800 is provided with a pair of opposing internal longitudinal grooves 812. The area radially outward of the terminus of a groove is machined away 816. Clockwise rotation of the driver causes the pins 854 to abut an adjacent portion of the fastener 800 and thus cause the fastener to rotate and advance in the forward direction 820. However, counter clockwise rotation of the driver 860 causes the pins 854 to rotate and thus depart from the groove or grooves 812 and come to rest in a position where withdrawal of the driver tip 852 is resisted by a portion of the fastener body. In this orientation, anti-clockwise rotation of the driver 860 acts to withdraw the fastener 800 (retrograde motion, i.e. in the direction opposition of arrow 820). Further, putting the driver into tension to assist in the withdrawal cannot disengage the driver from the fastener 800. A small clockwise rotation of the driver realigns the pins 854 with the channels 812 so that the tip 852 can be withdrawn from the fastener 800.

As shown in FIG. 8C, the driver tip 852 can be constructed by providing transverse passageways for receiving the pins 854. In this example, two pins extend through the entire diameter of the driver and beyond the outer surface to create four projections 854. Note that the configuration of the internal grooves 812 prevents the extreme distal tip 856 of the driver from making contact with the saddle 320. FIG. 8C also illustrates that by way of example only, and according to the present embodiment the radial extent of the pins 854 is below the root 832 of the cutting threads 830. By making the tip diameter of the pins 854 smaller than even the smallest diameter root 834, the insertion of the driver and its pins 854 is never resisted by bone material that may occupy the space between the threads 830.

As shown in FIG. 8D each pin 854 comes to rest, after the driver has been inserted and rotated counter clockwise into a transverse side channel 840. In the side channel (withdrawal position) it is preferred that the round pins 854 make surface contact 842 with the body of the fastener 800. This requires that the side walls 842 of the groove's side-channel have a generally semi circular configuration where they are contacted by a round pin. It will be appreciated that, in an alternative embodiments, other co-operating configurations can be used, for example substantially rectangular pin and a square set groove.

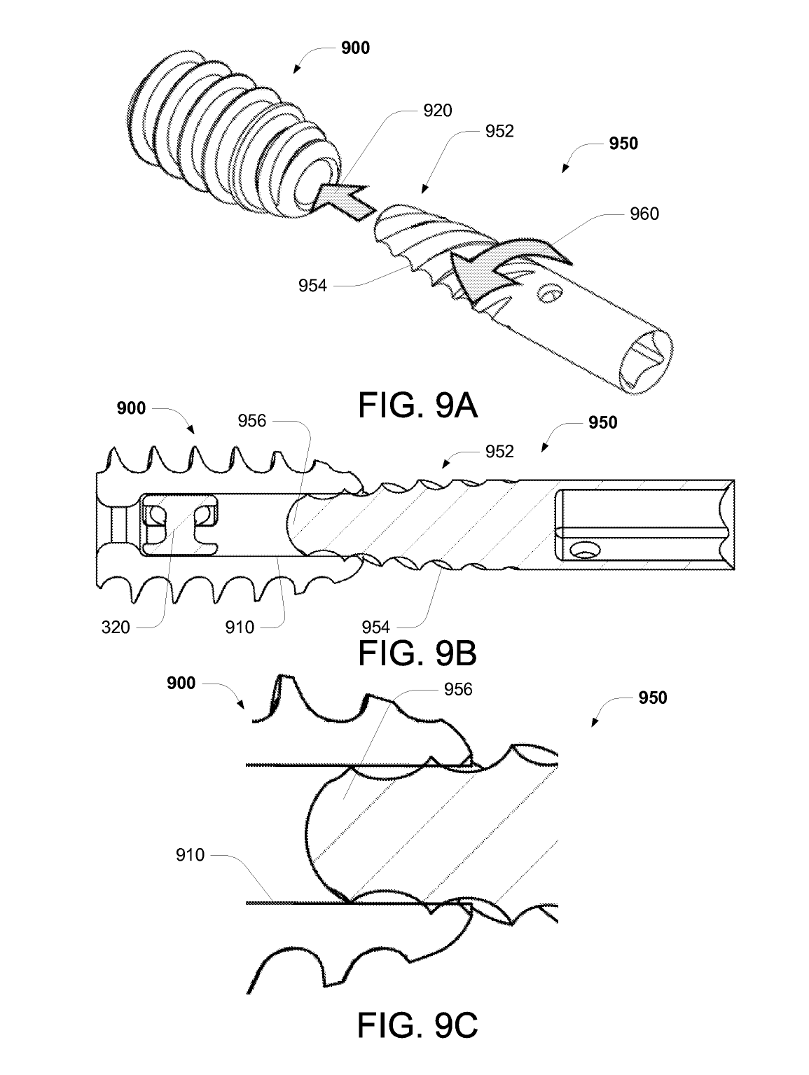

Another example of a driver is depicted in FIG. 9A and FIG. 9B. A retrograde fastener 900 has been configured to receive a specially adapted driver 950. In this example, the tip 952 of the driver is in the form of a fastener extractor. The tip has hardened, tapered, coarse threads 954 that are anti-clockwise. As shown in FIG. 9B and FIG. 9C, the extreme tip 956 of the driver can be inserted into the smooth interior bore 910 of the fastener 900. Anti-clockwise rotation causes the threads 954 to advance and cut into the bore 910. The fastener 900 is thus withdrawn through the bone tunnel with the anti-clockwise motion of the driver. The driver can be put into considerable tension without the threads 954 disengaging. Clockwise rotation of the driver causes the tip 956 to reverse of the bore 910 and thus causes disengagement of the driver with the fastener 900. It will be appreciated that for this type of driver, the threads 954 must be harder than the internal bore 910 of the fastener 900.

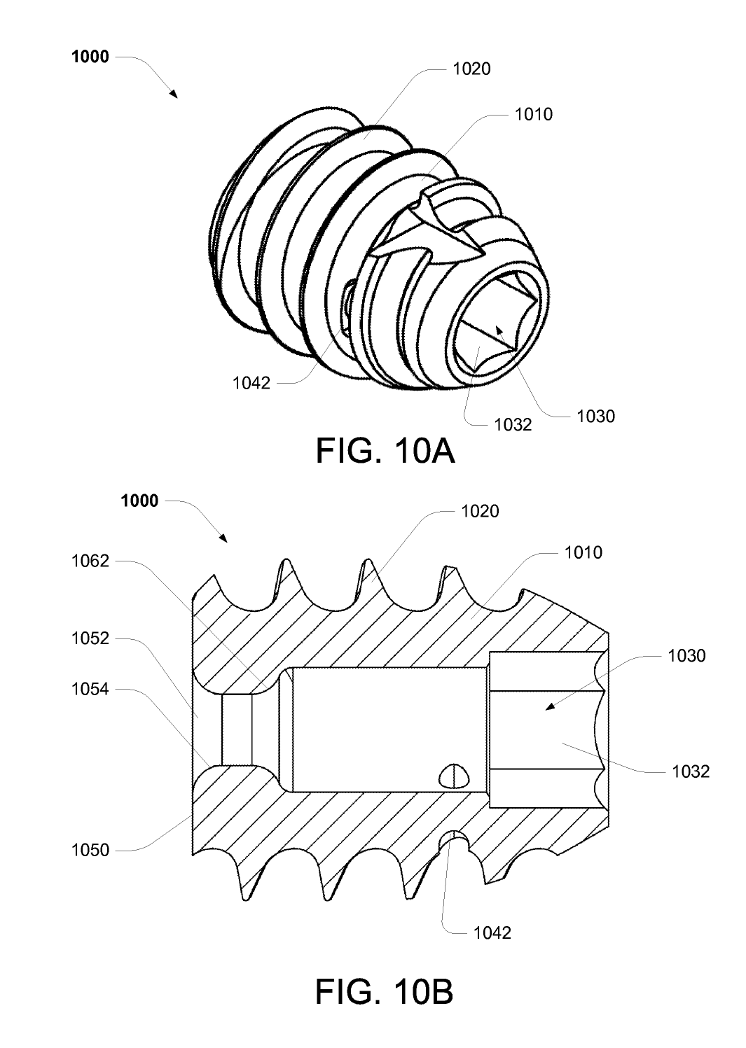

FIG. 10A and FIG. 10B show an embodiment screw fastener 1000. A first coupling element is shown at the proximal end of the body for coupling a driver tool.

This embodiment (similar to the embodiment screw fastener 100) comprises a relatively coarse threaded slightly tapered plug (or body) 1010 having the fastening characteristics of a bone screw. External screw threads 1020 are adapted to be self tapping into a tunnel pre-drilled through the tibia or femur. A central longitudinal bore or passageway 1030 extends through the fastener, from one end to the other. The proximal end 1032 of the passageway forms a socket for receiving a cooperating driver. It will be appreciated that the socket, and therefore the head of a corresponding driver, can comprise numerous configurations, including a inwardly scalloped hex socket. A receiving driver is adapted to accommodate the socket configuration. One or more transverse through openings 1042 can accommodate a loop of material such as polyethylene or polyester or other type of suture material without interfering with the operation of the fastener, the fastener's threads or the socket 1030.

As shown in FIG. 10B, the distal end 1050 of the fastener 1000 comprises a portal 1052 that leads into the central bore or through passageway 1030. The portal 1052 comprises a smoothly radiused rim and a smooth opening 1054 for receiving the loop or suture arrangement as herein described. A saddle (not shown) is adapted to rotate freely within the passageway and is restrained in its axial movement toward the portal 1052 by a necking down 1062 of the passageway adjacent to the portal 1052.

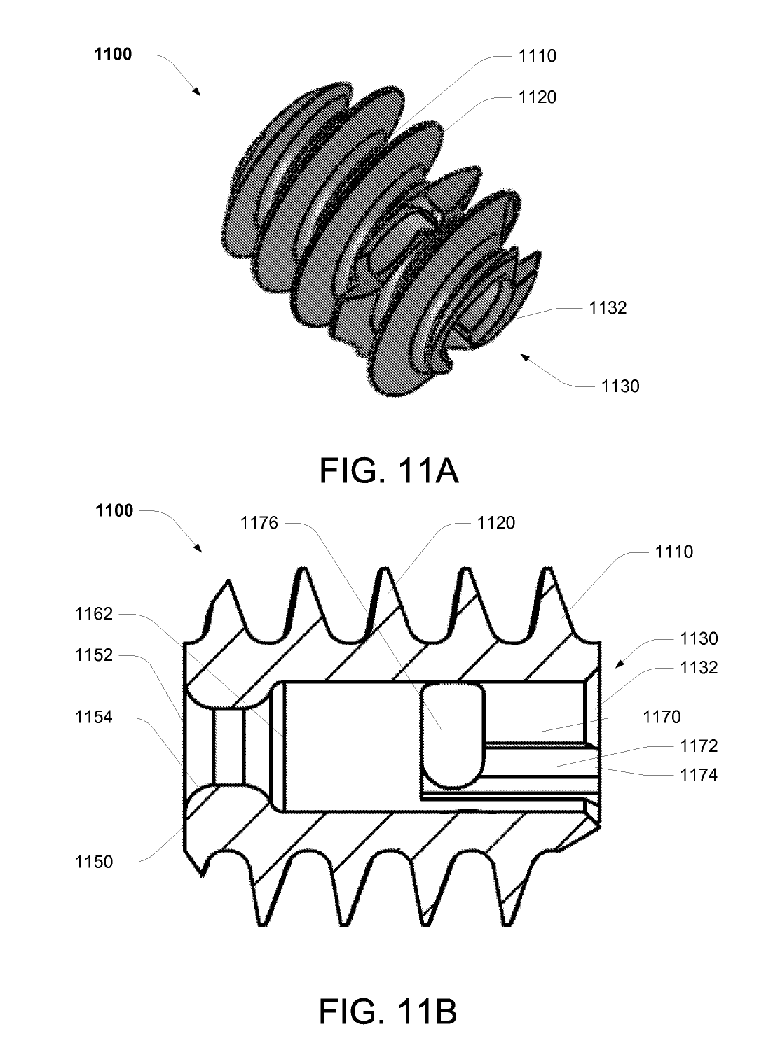

FIG. 11A and FIG. 11B show an embodiment screw fastener 1100. A first coupling element is shown at the proximal end of the body for coupling a driver tool.

This embodiment (similar to the embodiment screw fastener 100) comprises a relatively coarse threaded slightly tapered plug (or body) 1110 having the fastening characteristics of a bone screw. External screw threads 1120 are adapted to be self tapping into a tunnel pre-drilled through the tibia or femur. A central longitudinal bore or passageway 1130 extends through the fastener, from one end to the other. The proximal end 1132 of the passageway forms a socket for receiving a cooperating driver.

As shown in FIG. 11B, the distal end 1150 of the fastener 1100 comprises a portal 1052 that leads into the central bore or through passageway 1130. The portal 1052 comprises a smoothly radiused rim and a smooth opening 1154 for receiving the loop or suture arrangement as herein described. A saddle (not shown) is adapted to rotate freely within the passageway and is restrained in its axial movement toward the portal 1152 by a necking down 1162 of the passageway adjacent to the portal 1152.

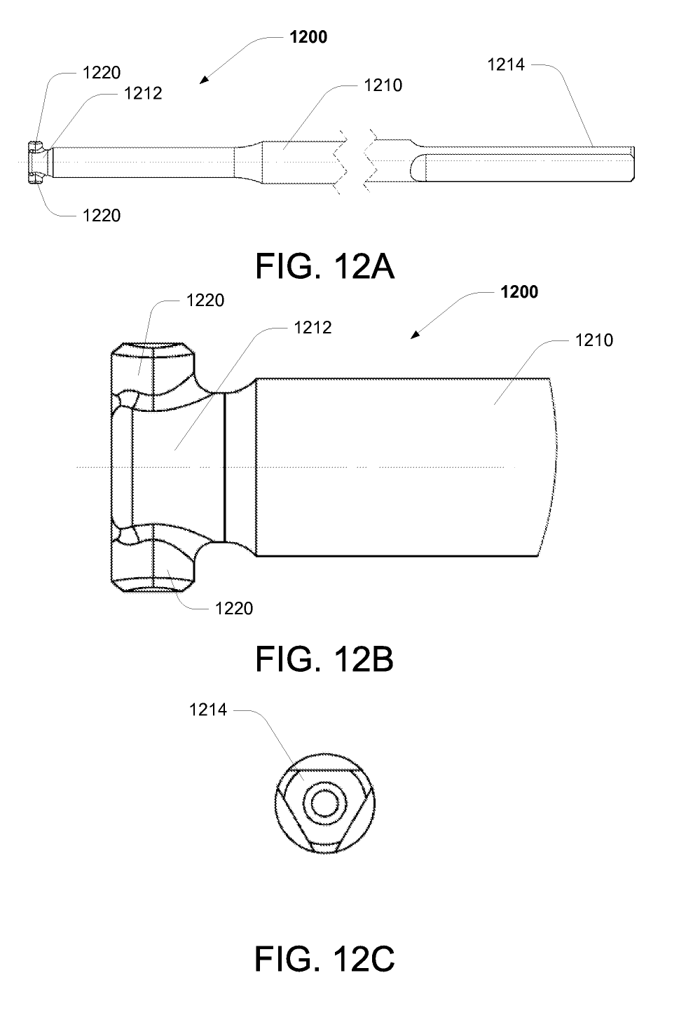

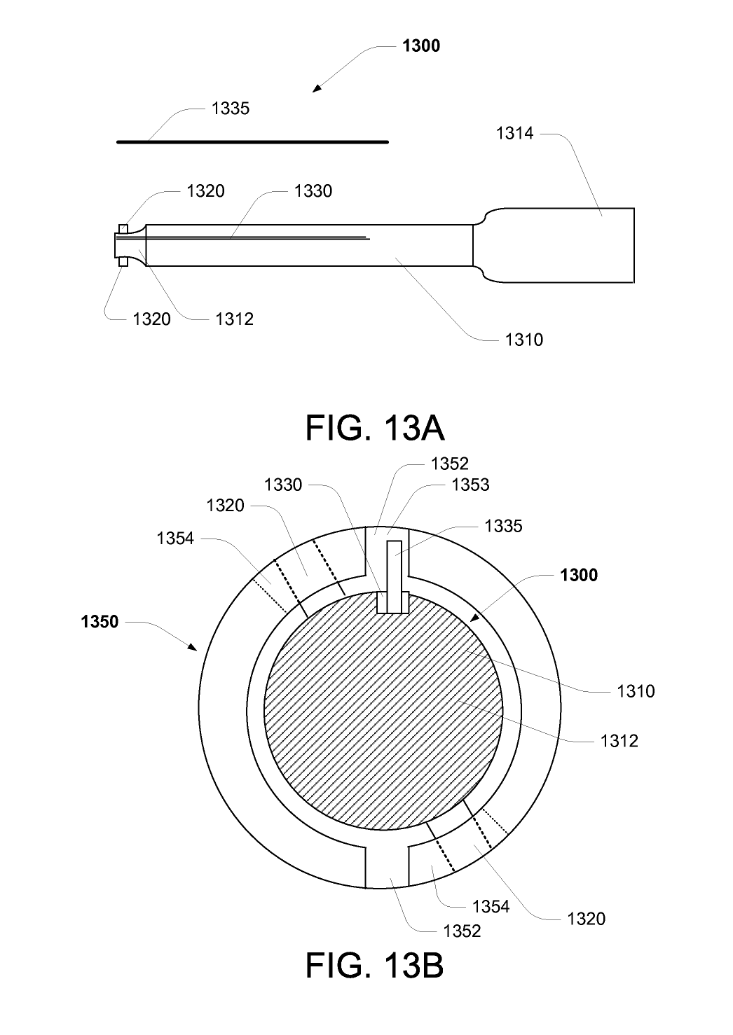

It will be appreciated that the socket, and therefore the head of a corresponding driver, can comprise numerous configurations, including a bayonet style connection. A receiving driver (as best shown in FIG. 12 and FIG. 13) is adapted to accommodate the socket configuration.

In this embodiment, a bayonet style connection 1170 can comprise a one or more longitudinal channels 1172 extending approximately a third to halfway down the periphery of longitudinal bore or passageway 1130, providing a relief groove that starts by intersection of the proximal rim 1174 of the fastener and terminates at a radially scribed passageway 1176.

By way of example only, when inserting a driver (not shown) into the socket 1130, the bayonet style connection 1170 enables a releasable coupling such that the screw fastener 1100 can be pulled though or to a tunnel in a bone. A pin on the driver engages and traverses the longitudinal channel 1172, such that upon full insertion of the driver, the driver can be axially rotated such that the pin sweeps the radially scribed passageway 1176.

The configuration of the internal channel (or grooves) are adapted to prevent the extreme distal tip of the driver from making contact with the saddle.

FIG. 12A and FIG. 12B show an embodiment driver (or adaptor) 1200 for using screw fastener (for example screw fastener 1100).

This embodiment driver (or adaptor) 1200 has an elongate shaft 1210, terminating at one end with a coupling element for engaging a socket of a screw fastener.

The driver 1200 comprises a generally cylindrical tip (distal tip) 1212 having one or more radially extending pins or projections 1220. In this example, the driver 1200 is provided with two oppositely directed radially extending pins on the tip 1212. It will be appreciated that other configurations are contemplated. In particular, in an embodiment only one pin or protrusion be provided.

In order that the fastener (for example screw fastener 1100, not shown) receive the tip of the driver 1212, the internal bore of the fastener is provided with a pair of opposing internal longitudinal channels or grooves. The area radially outward of the terminus of a groove is machined away. Clockwise rotation of the driver causes the pins 1220 to abut an adjacent portion of the fastener and thus cause the fastener to rotate and advance in the forward direction. Counter clockwise rotation of the driver 1200 causes the pins 1220 to rotate and thus depart from the longitudinal channel or groove and come to rest in a position where withdrawal of the driver tip is resisted by a portion of the fastener body. In this orientation, further anti-clockwise rotation of the driver 1200 acts to draw the fastener (retrograde motion) though a tunnel in a bone. Further, putting the driver into tension to assist in the drawing (or withdrawal) of the fastener through a tunnel cannot disengage the driver from the fastener. A small clockwise rotation of the driver realigns the pins 1220 with the channels so that the tip 1212 can be withdrawn from the fastener.

As shown in each pin comes to rest, after the driver has been inserted and rotated counter clockwise into a transverse side channel. In the side channel (withdrawal position) it is preferred that the round pins make surface contact with the body of the fastener. This requires that the side walls of the groove's side-channel have a generally semi circular configuration where they are contacted by a round pin. It will be appreciated that, in alternative embodiments, other co-operating configurations can be used, for example substantially rectangular pin and a square set groove.

In this example the driver 1200 comprises a generally triangular proximal tip 1214 for receiving a handle, a torque driver, or a second driver (for example a drill). It will be appreciated that, in alternative embodiments, other co-operating configurations can be used.

FIG. 13A and FIG. 13B show an alternative embodiment driver (or adaptor) 1200 for using screw fastener 1100.

Similar to the embodiment shown in FIG. 12A and FIG. 12B, this embodiment driver (or adaptor) 1300 has an elongate shaft 1310, terminating at one end with a coupling element for engaging a socket of a screw fastener (as best shown in FIG. 13B).

The driver 1300 comprises a generally cylindrical tip (distal tip) 1312 having one or more radially extending pins or projections 1320. Initial fastening of the driver to a screw fastener was outlined in the description referring to FIG. 12A and FIG. 12B.

Referring to FIG. 13B, in this embodiment, the internal bore of the fastener 1350 receives the tip of the driver 1312, whereby the pins 1320 are received by a pair of opposing internal longitudinal channels or grooves 1352. Counter clockwise rotation of the driver 1300 causes the pins 1320 to rotate and thus depart from the longitudinal channel or groove 1352 and sweep a respective radially scribed passageway 1354. The pins come to rest in a position where withdrawal of the driver tip is resisted by a portion of the fastener body.

In this example the driver 1200 further comprises a longitudinally extending channel 1330, such that when the driver is engaged with the screw fastener, the longitudinally extending channel 1330 of the driver aligns with an internal longitudinal channel 1352 of the screw fastener. An elongate rod or wire 1335 can be located within both channels 1330 and 1352 to restrict further relative rotation between the screw fastener and the driver. It will be appreciated that this configuration restricts the fastener from being able to fall off the driver. It will be further appreciated that, in an alternative embodiments, other locking configurations for restricting further relative rotation between the screw fastener and the driver can be used. Once the screw fastener is in place the thin wire can be removed to allow the driver to be decoupled from the screw fastener.

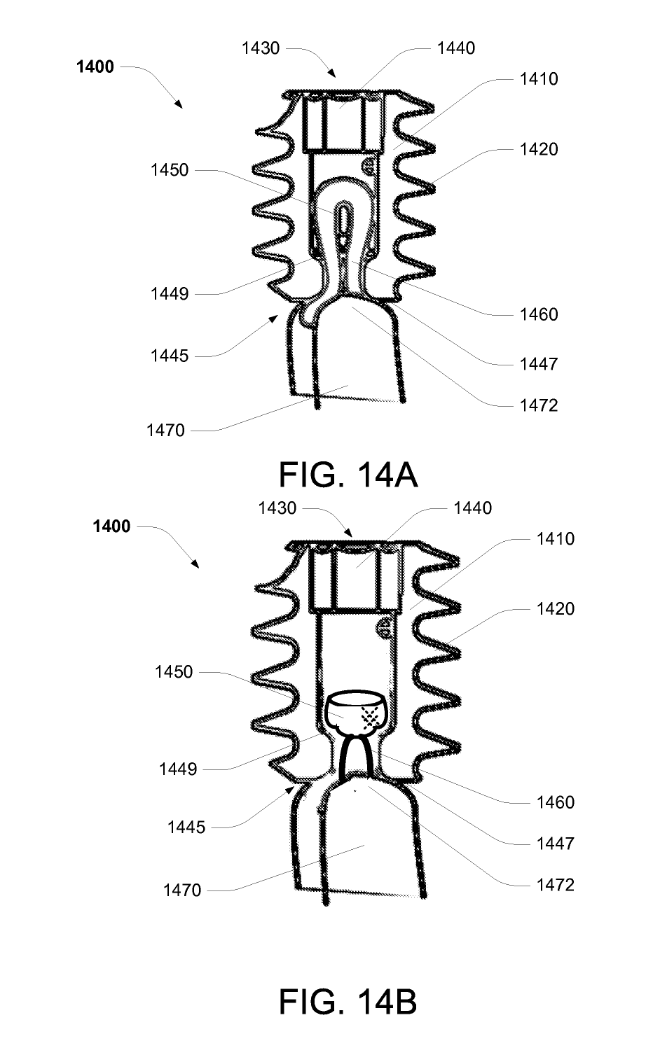

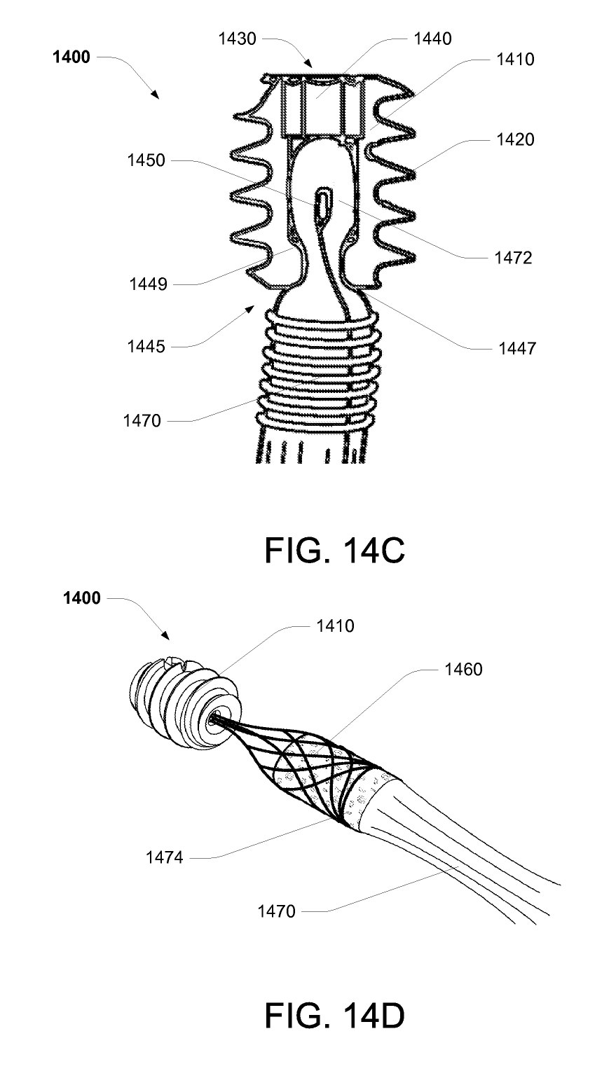

FIG. 14A through FIG. 14D show alternative structures fastening element for rotatably coupling (or attaching) a graft/tendons to a screw fastener. However, it will be appreciated that structures for rotatably coupling (or attaching) a graft are not limited to these particular embodiments. These embodiments shows alternative second coupling element for rotatable coupling of a first end of the graft with respect to the device.

The screw fastener includes a body 1410 having an external screw thread 1420. A central longitudinal bore or passageway 1430 extends through the fastener, from one end to the other. The proximal end 1440 of the passageway forms a socket for receiving a cooperating driver. The distal end 1445 of the screw fastener comprises a portal 1447 that leads into the central passageway 1430. A saddle 1450 is adapted to rotate freely within the passageway and is restrained in its axial movement toward the portal 1447 by a necking down 1449 of the passageway adjacent to the portal. The graft 1470 is rotatably couplable to the screw fastener.

It will be appreciated that a first end 1472 of replacement tendons or graft 1470 will be rotatably couplable to a screw fastener, and the second (other) end fixedly couplable to a bone. Then replacement tendons are provided by tendons that are looped around a coupling operatively associated with the screw fastener, the first end is defined by the portion of tendons/graft adjacent the screw fastener (when rotatably coupled) and the second end is defined by free ends of tendons/graft (or the other end).

FIG. 14A is a sectional view of an embodiment fastener 1400 made in accordance with the teachings of the present invention.

In this embodiment, a flexible loop element 1460 is located around the saddle 1450. The loop extends below the portal 1447, such that the graft (or tendons) can pass though and/or be coupled to the loop. The free ends of graft (or tendons) define the second end of the graft. A saddle 1450 is adapted to rotate freely within the passageway thereby providing a rotatable coupling between the screw fastener and the graft 1470. The saddle 1450 and loop element 1460 can be located in the passageway 1430 by passing them though the opening at the proximal end 1440.

FIG. 14B is a sectional view of an embodiment fastener made in accordance with the teachings of the present invention.

In this embodiment, a flexible loop 1460 is integrally formed with the saddle 1450, for example in the form of an expansion of the looped material. The loop extends below the portal 1047, such that the graft (or tendons)can pass though and/or be coupled to the loop. The free ends of tendons define the second end of the graft. A saddle 1450 is adapted to rotate freely within the passageway thereby providing a rotatable coupling between the screw fastener and the graft 1470. The saddle 1450 and loop element 1460 can be located in the passageway 1430 by passing them though the opening at the proximal end 1040.

By way of example only, the combination saddle and loop can comprise a loop having an expanded substantially non-compressible end, such that the material and configuration were sufficiently non-compressible that the expanded end would not pass though the necking down 1449 of the passageway 1430.

By way of example only an expansion the looped material can be in the form of a tight weave, a specialised knot or treatment of the loop material in such a way that it is restricted from pass though the necking down 1449 of the passageway 1430, but still enabled to rotate within the passageway 1430

FIG. 14C is a sectional view of an embodiment fastener made in accordance with the teachings of the present invention.

In this embodiment, the graft 1470 is received by the portal 1047 and extend around the saddle 1450. The free ends of tendons define the second end of the graft. A saddle 1450 is adapted to rotate freely within the passageway thereby providing a rotatable coupling between the screw fastener and the graft 1470. The saddle 1450 and tendons /graft 1470 can be located in the passageway 1430 by passing them though the opening at the proximal end 1040.

By way of example an artificial graft can be combined from multiple artificial tendon strands that extend around the saddle and are braded in situ, for providing a rotatable coupling to the screw fastener.

FIG. 14D is a sectional view of an embodiment fastener made in accordance with the teachings of the present invention.

In this embodiment, when performing a bone ligament reconstruction, a webbing configuration, grabbing suture configuration, trap type configuration, or the like 1460 can be used to couple bone 1472 at the first end of the transplanted tendon/graft 1470. The configuration can be extend around the saddle, thereby providing a rotatable coupling to the screw fastener

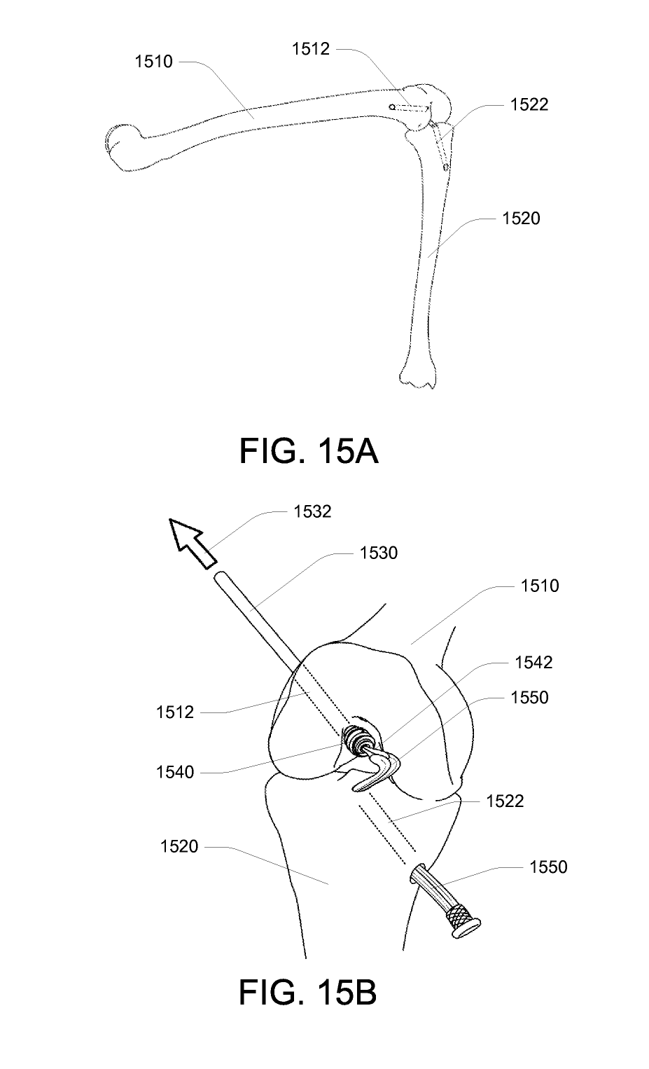

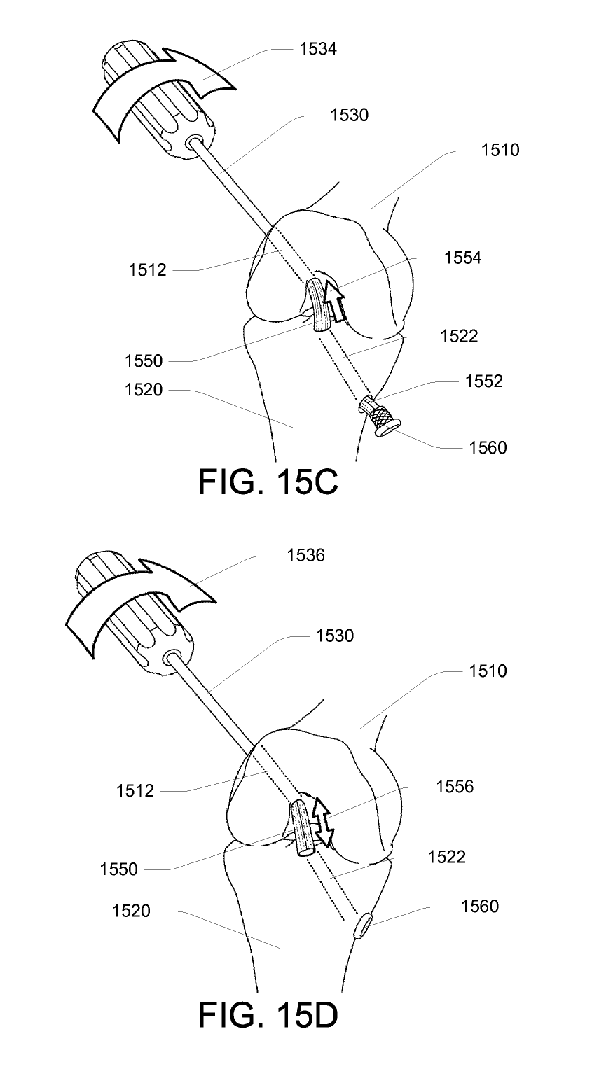

Referring to FIG. 15A though 15D, a method of using a fastener in an ACL reconstruction is disclosed.

FIG. 15A shows holes are first drilled in the femur 1510 and tibia 1520 to form a femoral tunnel 1512 and tibial tunnel 1522.

FIG. 15B shows that a driver 1530 can be passed through either tunnel (1512 or 1522), in any direction. The screw fastener 1540 can be prefixed on the driver or placed in position once the driver is passed through the holes. The tendons/graft 1550 can be rotatably coupled to the screw fastener 1540, for example by passing the to the tendons/graft through a loop 1542 located about a saddle (not shown). The driver can then be pulled up though the tunnel (as indicated by arrow 1532) such that the screw fastener engages the bone.

FIG. 15C shows that as the driver 1530 and coupled screw fastener (not shown) can be rotated (as indicated by arrow 1534) with respect to the respective bone. This causes the screw fastener to threadedly engage the bone and thereby draw the tendons/graft 1550 up though the tunnel (as indicated by arrow 1554). In this embodiment, the saddle rotates within the screw as the graft is being pulled up so as to not twist the graft. The opposite end of the graft 1552 is fixed relative to the tibia 1520. By way of example only, an end plug 1560 can be fixed opposite end of the graft 1552, such that drawing up the graft bring the end plug 1560 into abutting engagement (or seated) with the tibia 1520. It will be appreciated that the opposite end 1552 of the tendons/graft 1550 to the screw (or the tendons/graft free end) can be fixed in any surgically suitable manner.

FIG. 15D shows that once the opposite end 1552 of the graft is fixed relative to the tibia 1520, the driver 1530 and coupled screw fastener (not shown) can be rotated in a clockwise or anticlockwise direction with respect to the respective bone (as indicated by arrow 1536). This causes the screw fastener to threadedly engage the bone and thereby increases or decreases tension applied to tendons/graft 1550 (as indicated by arrow 1556). A torque driver can be used to fine tuned the tension applied to the tendons/graft 1550.

The driver 1530 can then be detached from the screw fastener 1540.

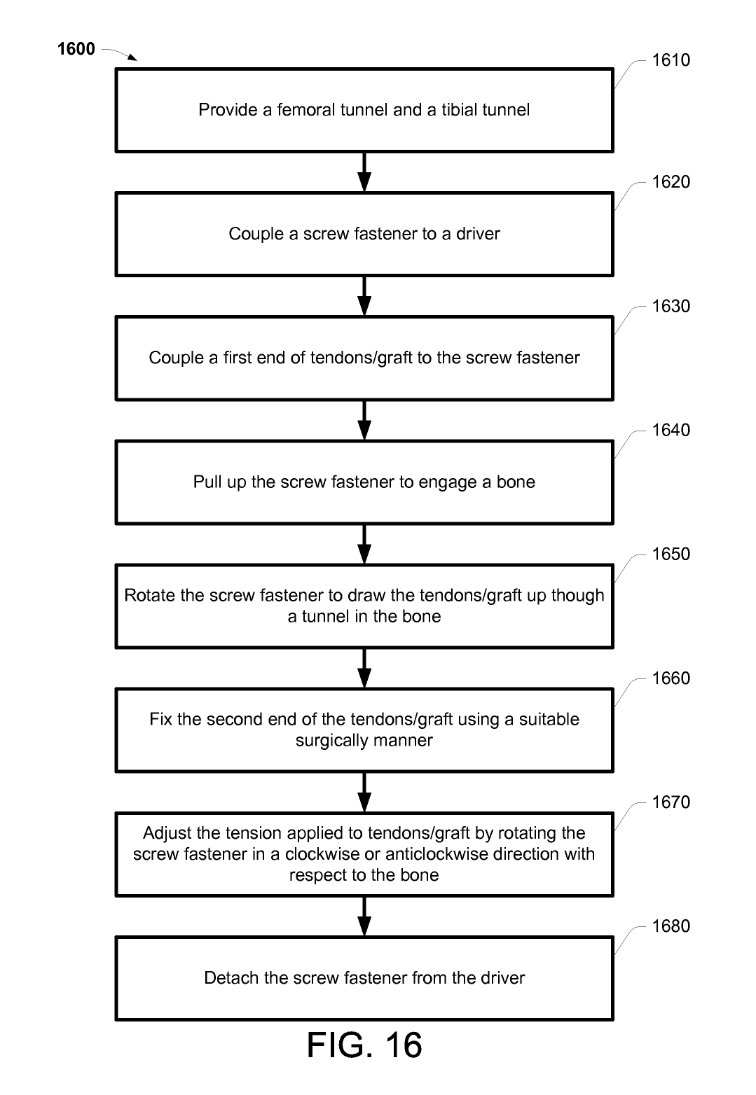

Referring to FIG. 16, a method 1600 of using a fastener in an ACL reconstruction can comprise the steps of:
STEP 1610: providing a femoral tunnel and a tibial tunnel in the femur and tibia respectively;
STEP 1620: coupling a screw fastener to a driver passed through either tunnel;
STEP 1630: rotatably coupling a graft to the screw fastener;
STEP 1640: pulling up the screw fastener engages a bone;
STEP 1650: rotating the screw fastener, by rotating the driver, causing the screw fastener to threadedly engage the bone and thereby draw the graft up though the tunnel.
STEP 1660: fixing the opposite end of the graft to the screw (or the tendons/graft free end) to a bone using a suitable surgical manner;
STEP 1670: with the opposite end of the graft fixed relative to a bone, the screw fastener (and coupled driver) can be rotated in a clockwise or anticlockwise direction with respect to a respective bone to thereby increase or decrease tension applied to tendons/graft.
STEP 1680: detaching the screw fastener from the driver.

In this embodiment, a saddle rotates within the screw as the tendons/graft is being pulled up so as to not twist the graft. The opposite end of the graft can be fixed relative to the tibia. By way of example only, an end plug can be fixed to the opposite end of the graft, such that drawing up the graft bring the end plug into abutting engagement (or seated) with the tibia. It will be appreciated that the opposite end of the graft to the screw (or the tendons/graft free end) can be fixed in any surgically suitable manner.

While the present invention has been disclosed with reference to particular details of construction, these should be understood as having been provided by way of example and not as limitations to the scope or spirit of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

Similarly, it is to be noticed that the term coupled, when used in the claims, should not be interpreted as being limitative to direct connections only. The terms “coupled” and “connected”, along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical, or that two or more elements are not in direct contact with each other but yet still co-operate or interact with each other.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

As used herein, unless otherwise specified the use of terms “horizontal”, “vertical”, “left”, “right”, “up” and “down”, as well as adjectival and adverbial derivatives thereof (e.g., “horizontally”, “rightwardly”, “upwardly”, etc.), simply refer to the orientation of the illustrated structure as the particular drawing figure faces the reader, or with reference to the orientation of the structure during nominal use, as appropriate. Similarly, the terms “inwardly” and “outwardly” generally refer to the orientation of a surface relative to its axis of elongation, or axis of rotation, as appropriate.

Similarly it should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth.

However, it is understood that embodiments of the invention may be practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

1. A surgical screw fastener device for pulling a graft through a first tunnel defined in a first bone, the surgical screw fastener including:
a body having a proximal end and a distal end;
an exterior screw thread located around the body for threadedly engaging a wall of the first tunnel;
a first coupling element at the proximal end of the body, the first coupling element adapted to couple a driver tool; and
a second coupling element for rotatable coupling a first end of the graft with respect to the device.

2. The device according to claim 1, wherein:
the device defines a through passage having an aperture at the distal end and the proximal end.

3. The device according to claim 2, wherein:
the first coupling element is a female socket at the proximal end of the body.

4. The device according to claim 3, wherein:
the female socket is defined by the through passage having an aperture at the proximal end.

5. The device according to claim 3, wherein:
the first coupling element is a releasable coupling element for securely coupling the device to the driver tool.

6. The device according to claim 4,
wherein the a second coupling element comprises a saddle element that is locatable within the through passage and proximal to the distal end; the second coupling element being rotatable with the through passage.

7. The device according to claim 6, wherein:
the saddle element is locatable within the through passage by being passed through an aperture at the proximal end defined by the passage.

8. The device according to claim 7, wherein:
the saddle element is adapted to rotate freely within the passageway and is restrained in its axial movement toward the distal end by a necking down of the passageway.

9. The device according to claim 8, wherein:
the a second coupling element further comprises a fastening element coupled to the saddle element and adapted to retain a first end of the graft.

10. The device according to claim 9, wherein the fastening element includes any one or more of the set comprising:
a loop element;
a net element.

11. The device according to claim 9, wherein the fastening element is integrally formed with the saddle element.

12. The device according to claim 9, wherein:
the fastening element is integrally formed with an artificial graft.

13. The device according to claim 9, wherein:
the fastening element is constructed of a flexible material.

14. The device according to claim 9, wherein:
selective clockwise or anticlockwise rotation of the body, while threadedly engaging the wall of the first tunnel, can respectively increase or decrease tension applied to the graft.

15. The device according to claim 9, wherein:
selective anticlockwise or clockwise rotation of the body, while threadedly engaging the wall of the first tunnel, can respectively increase or decrease tension applied to the graft.

16. (canceled)

17. A method of using a surgical screw fastener device for pulling a graft through a first tunnel defined in a first bone, the method comprising the steps of:
(a) providing a screw fastener device;
(b) coupling the screw fastener to a driver tool passed through the first tunnel;
(c) rotatably coupling a first end of the graft to the device;
(d) rotating the screw fastener, by rotating the driver tool, causing the screw fastener to threadedly engage the bone and thereby draw the graft up though the tunnel;
(e) with other end of the graft fixed in location, the screw fastener can be rotated with respect to the bone to thereby set a tension applied to the graft; and
(f) detaching the screw fastener from the driver tool.

18. The method according to claim 17, wherein the screw fastener device comprises:
a body having a proximal end and a distal end;
an exterior screw thread located around the body for threadedly engaging a wall of the first tunnel;
a first coupling element at the proximal end of the body, the first coupling element adapted to couple a driver tool; and
a second coupling element for rotatable coupling a first end of the graft with respect to the device.

19. The method according to claim 17, wherein fixing the other end of the graft includes abutment of an end plug fixed to the other end of the graft as a result of the device drawing up the graft.

20. (canceled)
